(19) United States
(12) Patent Application Publication
(10) Pub. No.: US 2010/0094142 A1
(43) Pub. Date: Apr. 15, 2010

(54) Display Module of LED Light Pillar of Hemomanometer and the Hemomanometer with LED Light Pillar

(75) Inventors: BAOSHAN YU, Hangzhou (CN); GONGYI LIN, Hangzhou (CN)

Correspondence Address: GLOBAL IP SERVICES, 7285 W. Eagle Court, Winton, CA, 95388, US

(73) Assignee: BAOSHAN YU, Hangzhou (CN)

(21) Appl. No.: 12/573,375

(22) Filed: Oct. 5, 2009

(30) Foreign Application Priority Data
Oct. 10, 2008 (CN) 200820153954.6
Dec. 31, 2008 (CN) 200820208889.2
Feb. 16, 2009 (CN) 200920066710.9

(51) Int. Cl. A61B 5/02; G01D 11/28

(52) U.S. Cl. 600/485; 362/29

(57) ABSTRACT

The present invention relates to an instrument for measuring blood pressure. More particularly, the present invention relates to a display module of LED light pillar of hemomanometer. Which comprises a shell (1) containing a LED printed circuit board (2) that has a multiple of pins (21) at one side and corresponding multiple of LED tubes (22) at the other side; a cover plate (3) provided with a multiple of through holes (31); a driving circuit board (4) provided at the external side of cover plate (3); each pin (21) of the LED printed circuit board (2) passes each through hole (31) of the cover plate (3) correspondingly and links to driving circuit board (4) and the cover plate (3) and shell (1) jointly encapsulate the LED printed circuit board (2); a top of the shell (1) is transparent in on which a linear grating (5) is provided. The object of the present invention is to encapsulate LED printed circuit board in its shell without requiring epoxy resin.

### CROSS REFERENCE TO THE RELATED PATENT APPLICATIONS

This patent application claims the priorities of the Chinese patent application No. 200820153954.6 filed on Oct. 10, 2008, Chinese patent application No. 200820208889.2 filed on Dec. 31, 2008 and Chinese patent application No. 200920066710.9 filed on Feb. 16, 2009, which applications are incorporated herein by reference.

### BACKGROUND OF THE INVENTION

1. Field of the Invention

The present invention relates to an instrument for measuring blood pressure. More particularly, the present invention relates to a display module of LED light pillar of hemomanometer.

2. Related Prior Art

The existing hemomanometer with LED light pillar has the following technical shortcomings:

1. LED printed circuit board of display module of LED light pillar in existing hemomanometer is encapsulated to its shell with epoxy resin. However, epoxy resin is subject to clinging to the electronic components of LED printed circuit board so as to affect product quality.

2. When the display panel of existing hemomanometer is opened, the measuring instrument cannot be automatically turned on. Pressing power ON/OFF button is needed. Pressing power ON/OFF button once again before closing the display panel is also needed. There has been not yet a hemomanometer that can be automatically turned on when its display panel is opened and turned off when the display panel is closed.

### SUMMARY OF THE INVENTION

The first object of the present invention is to encapsulate LED printed circuit board in its shell without requiring epoxy resin.

The second object of the present invention is to automatically turn on the hemomanometer when its display panel is opened and turn off the hemomanometer when its display panel is closed.

The first object which will become apparent from the discussion the follows, are achieved, according to the present invention, by providing a display module of LED light pillar of hemomanometer which comprising:

a shell (1) containing a LED printed circuit board (2) that has a multiple of pins (21) at one side and corresponding multiple of LED tubes (22) at the other side,

a cover plate (3) provided with a multiple of through holes (31),

a driving circuit board (4) provided at the external side of cover plate (3),

each pin (21) of the LED printed circuit board (2) passes each through hole (31) of the cover plate (3) correspondingly and links to driving circuit board (4), and the cover plate (3) and shell (1) jointly encapsulate the LED printed circuit board (2).

a top of the shell (1) is transparent in on which a linear grating (5) is provided.

The present invention, fixing the LED printed circuit board (2) in the shell (1) does not need epoxy resin but need adding a cover plate (3) in which a multiple of through holes (31) are provided. Each pin (21) of LED printed circuit board (2) passes each through holes (31) of the cover plate (3) and links to driving circuit board (4), and the cover plate (3) and the shell (1) jointly encapsulate the LED printed circuit board (2).

Preferably, the linear grating (5) being parallel to a longer side (11) of the shell (1) is integrally provided with the shell (3), or is bonded to an outer or inner surface of the shell (3).

Grating (5) is integrally provided with the shell (3), namely grating (5) is engraved at the transparent outer surface of the shell (3). Of course, the grating (5) can also be engraved at the transparent inner surface of the shell (3) or an individual grating can be bonded to the outer surface of transparent outer surface of the shell (3).

Preferably, the LED tubes are #301, #151 or $76 LED tubes (22).

The measuring range of hemomanometer is 0-300 mmHg, in accordance with practice and precision of measurement, when every 1 mmHg needs 1 LED tube (22) to display, 301 LED tubes are needed then, including 0 mmHg. When every 2 mmHg need 1 LED tube (22) to display, 151 LED tubes are needed then. When every 4 mmHg need 1 LED tube (22) to display, 76 LED tubes are needed then.

Preferably the LED tubes are #300, #150 or #75 LED tubes (22).

According to practice sometimes, if no LED tube is needed at 0 mmHg, then when every 1 mmHg needs 1 LED tube (22) to display, only 300 LED tubes are needed, excluding 0 mmHg. When every 2 mmHg need 1 LED tube (22) to display, only 150 LED tubes are needed then. When every 4 mmHg need 1 LED tube (22) to display, 75 LED tubes are needed then.

Preferably, one end of each through hole (31) is a seal (311) and the other end is a guide (312), diameters of the seal (311) and the pin (21) are identical, while a diameter of the conic guide (312) is bigger than that of the seal (311).

Seal (311) can closely coat pins to cause sealing effect. Conic guide facilitates intensive multiple pins inserting in through holes precisely and simultaneously.

Preferably, a taper of the guide (312) is 30-90 degrees.

Preferably, a boss (34) is provided under the cover plate (3) and is coupled with the shell (1) in the way of interference fit, ribbings (13) are provided at an external side of boss (34).

The interference fit between boss (34) and shell (1) ensures cover plate (3) to firmly insert in shell (1). Laser is used to melt ribbings (13) at outer side of boss (34) and glue to the encapsulating surface (12) of shell (1) to obtain a better sealing.

The second object which will become apparent from the discussion the follows, are achieved, according to the present invention, by providing the Hemomanometer with LED Light Pillar which comprises:

a soleplate (103) and two side plates (104, 105) jointly forming a cavity to hold blood pressure instrument;

a cover plate (101), hinged within the two side plate (104, 105), provided with the display module of LED light Pillar of hemomanometer (102) described in claim 1 or claim 5;

a trigger switch (205), provided on the left side plate (104), comprising a trigger tab (204) and a track groove (201) on the left, a cam (202), provided on the cover plate (101) by an axis (203), when sliding in the track groove (201), the cam being capable to engage or disengage the trigger tab (204) set on the trigger switch (205), when the cover plate (101) is completely opened, the cam (202) will engages the trigger tab (204) to turn on power, and when the cover plate (101) is closed, the cam (202) will disengages the trigger tab (204) to turn off power.

To use the hemomanometer, open its cover plate (101) to allow cam (202) to move from the lower end to upper end and the cam (202) will engage trigger tab (204) to turn on power. When the cover plate (101) is closed, the cam (202) will move from the upper end to the lower end and disengage trigger (204). The trigger (204) will be driven by spring and reset to its original position so as to turn off power automatically. Thus, neglect of turning off power after using the hemomanometer—unconventional consumption of battery—can be avoided.

### BRIEF DESCRIPTION OF THE DRAWINGS

FIG. 1 is a sectional view showing the encapsulated structure according to embodiment 1;

FIG. 2 is a sectional view before the encapsulation according to embodiment 1;

FIG. 3 is a sectional view along A-A line of FIG. 1;

FIG. 4 is a sectional view of cover plate in FIG. 3;

FIG. 5 is a sectional view of LED printed circuit board;

FIG. 6 is a view of grating structure in un-energized condition;

FIG. 7 is a view of turning from point source of light to linear source of light in energized condition.

FIG. 8 is a sectional view of embodiment 2;

FIG. 9 is a left view of FIG. 8;

FIG. 10 is a detail view of FIG. 9 when cover plate is closed;

FIG. 11 is a detail view of FIG. 9 when cover plate is opened;

### DESCRIPTION OF THE PREFERRED EMBODIMENTS

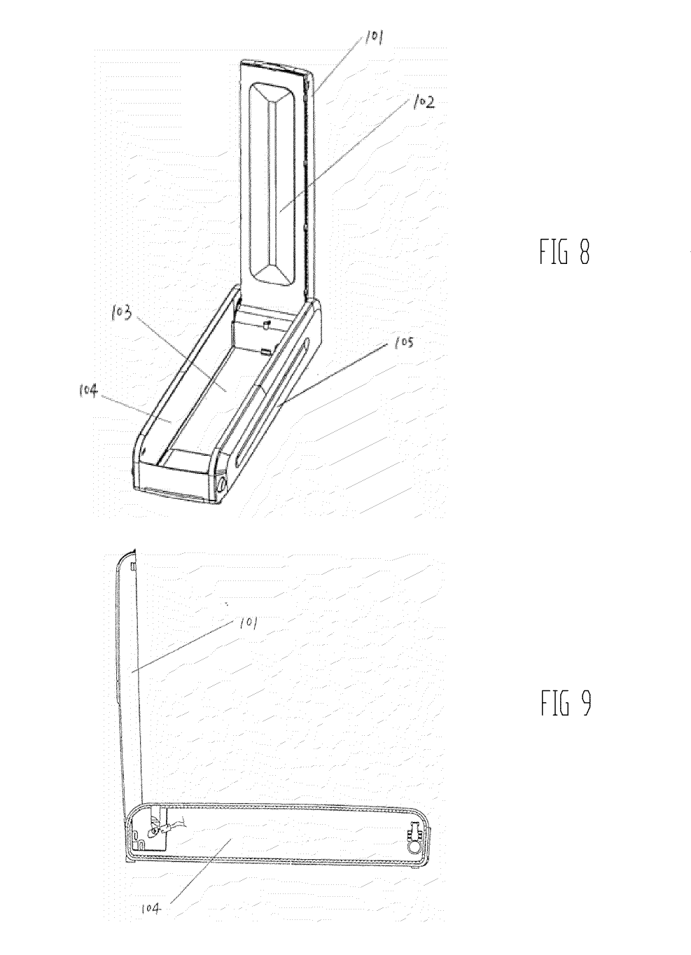

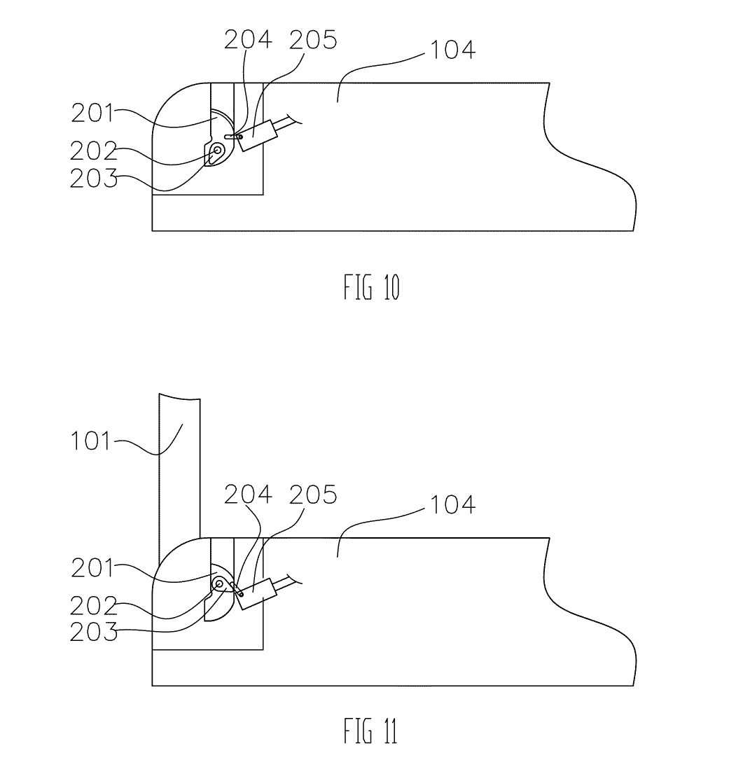

The preferred embodiment of the present invention will now be described with reference to FIGS. 1-11 of the drawings. Identical elements in the various figures are identified with the same reference numerals.

### Embodiment 1

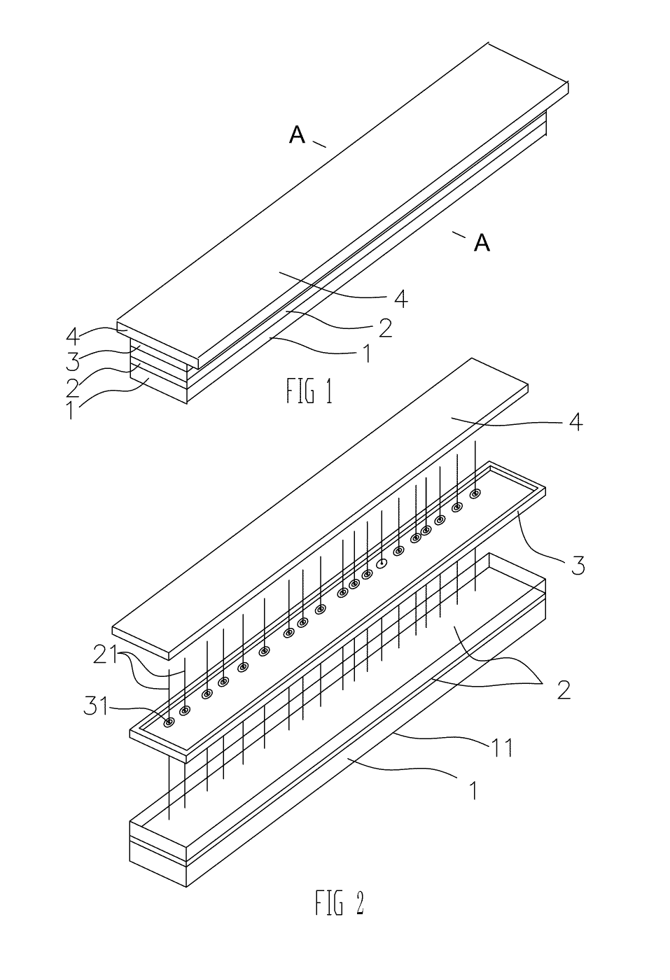

As shown in FIGS. 1 and 2, a display module of LED light pillar of hemomanometer comprising:

a shell 1 containing a LED printed circuit board 2 that has 301 pins 21 at one side and corresponding LED tubes 22 at the other side;

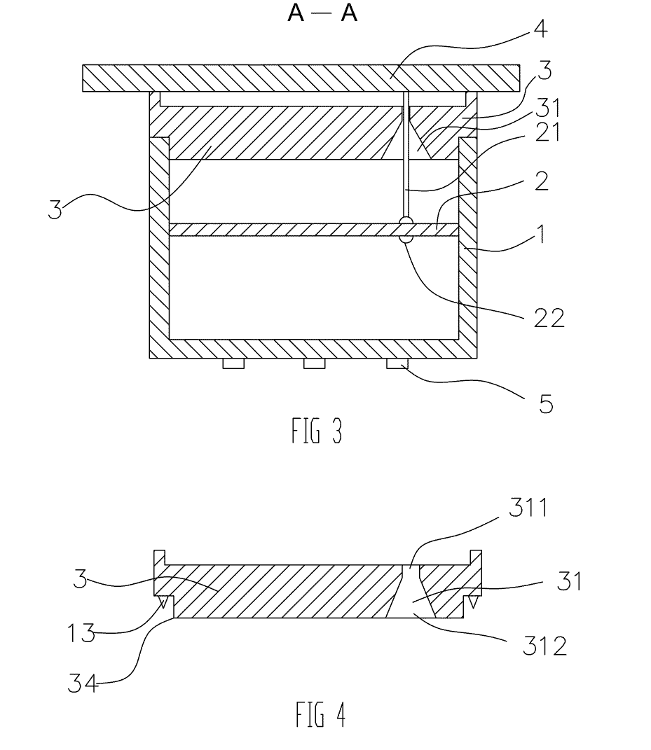

a cover plate 3 provided with 301 holes 31 as shown in FIGS. 3, 4 and 5, one end of each through hole 31 is a seal 311 and the other end is a guide 312, diameters of the seal 311 and the pin 21 are identical, while a diameter of the conic guide 312 is bigger than that of the seal 311, a. taper of the guide 312 is 45 degrees. A boss 34 is provided under the cover plate 3 and is coupled with the shell 1 in the way of interference fit, ribbings 13 are provided at an external side of boss 34;

a driving circuit board 4 provided at an external side of the cover plate 3;

Each pin 21 of the LED printed circuit board 2 passes each through holes 31 of the cover plate 3 and links to driving circuit board 4, and the cover plate 3 and shell 1 jointly encapsulate the LED printed circuit board 2.

Shell 1 is transparent in one side and in which a linear grating 5 is provided.

The linear grating 5 being parallel to the a longer side 11 of the shell 1, as shown in FIG. 7, and is integrally provided with the shell 3, namely grating is engraved at the transparent outer surface of the shell 3.

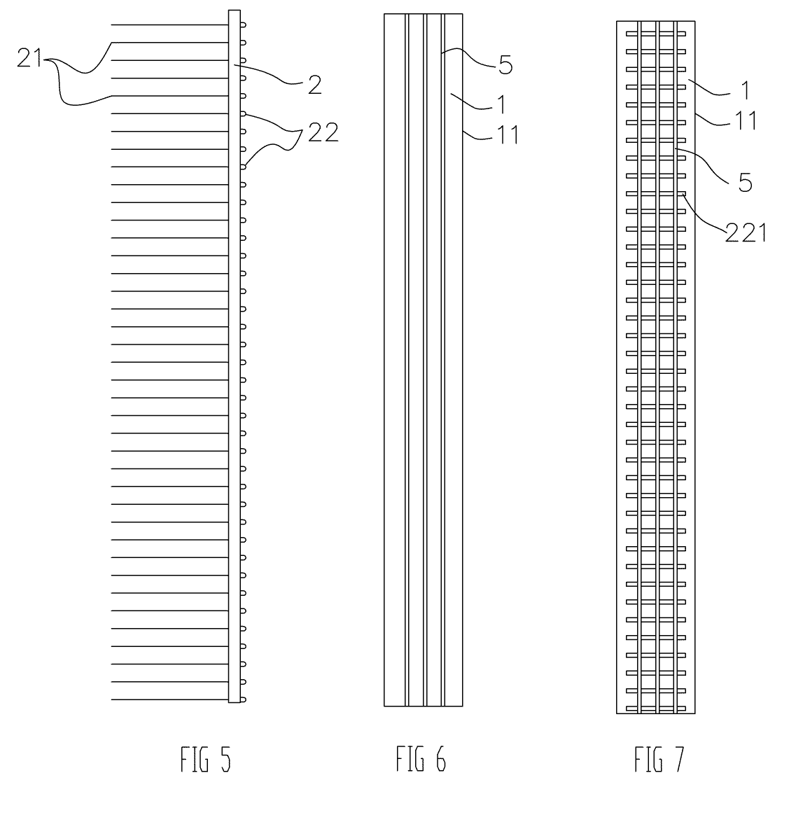

As shown in FIGS. 6 and 7, the LED tubes are #301, in the measuring range of 0-300 mmHg including 0 mmHg, a LED tube 22 is installed at the space of every 1 mmHg. When all 301 LED tubes emit light, grating effect will turn point source of light to linear source of light. The linear source of light corresponds to the scale of blood pressure for the convenience of reading.

### Embodiment 2

The Hemomanometer with LED Light Pillar comprising:

a soleplate 103 and two side plates 104, 105 jointly forming a cavity to hold blood pressure instrument;

a cover plate 101, hinged within the two side plates 104, 105, provided with the display module of LED light Pillar of hemomanometer 102 described in embodiment 1.

A trigger switch 205, provided on left side plate 104, comprising a trigger tab 204 and a track groove 201 on the left, a cam 202, provided on the cover plate 101 by an axis 203, when sliding in the track groove 201, the cam 202 capable to engage or disengage the trigger tab 204 set on the trigger switch 205, when the cover plate 101 is completely opened, the cam 202 engages the trigger tab 204 to turn on power, and when the cover plate 101 is closed, the cam 202 disengages the trigger tab 204 to turn off power.

1. A display module of LED light pillar of hemomanometer comprising:
a shell (1) containing a LED printed circuit board (2) that has a multiple of pins (21) at one side and corresponding multiple of LED tubes (22) at the other side,
a cover plate (3) provided with a multiple of through holes (31),
a driving circuit board (4) provided at an external side of the cover plate (3)
each pin (21) of the LED printed circuit board (2) passes each through hole (31) of the cover plate (3) correspondingly and links to driving circuit board (4), and the cover plate (3) and shell (1) jointly encapsulate the LED printed circuit board (2),
a top of the shell (1) is transparent and on which a linear grating (5) is provided.

2. The display module of LED light pillar of hemomanometer of claim 1, wherein the linear grating (5) being parallel to a longer side (11) of the shell (1) is integrally provided with the shell (3), or is bonded to an outer or inner surface of the shell (3).

3. The display module of LED light pillar of hemomanometer of claim 1, wherein the LED tubes are #301, #151 or #76 LED tubes (22).

4. The display module of LED light pillar of hemomanometer of claim 1, wherein the LED tubes are #300, #150 or #75 LED tubes.

5. The display module of LED light pillar of hemomanometer of claim 1, wherein one end of each through hole (31) is a seal (311) and the other end is a guide (312), diameters of the seal (311) and the pin (21) are identical, while a diameter of the conic guide (312) is bigger than that of the seal (311).

6. The display module of LED light pillar of hemomanometer of claim 5, wherein a taper of the guide (312) is 30-90 degrees.

7. The display module of LED light pillar of hemomanometer of claim 1, a boss (34) is provided under the cover plate (3) and is coupled with the shell (1) in the way of interference fit, ribbings (13) are provided at an external side of the boss (34).

8. A hemomanometer with LED Light Pillar comprising:
a soleplate (103) and two side plates (104, 105) jointly forming a cavity to hold blood pressure instrument;
a cover plate (101) hinged within the two side plates (104, 105), provided with the display module of LED light pillar of hemomanometer (102) described in claim 1;
a trigger switch (205) provided on the left side plate (104) comprising a trigger tab (204) and a track groove (201) on the left, a cam (202) provided on the cover plate (101) by an axis (203), when sliding in the track groove (201), the cam being capable to engage or disengage the trigger tab (204) set on the trigger switch (205), when the cover plate (101) is completely opened, the cam (202) engages the trigger tab (204) to turn on power, and when the cover plate (101) is closed, the cam (202) disengages the trigger tab (204) to turn off power.
